(19) United States
(12) Patent Application Publication
(10) Pub. No.: US 2021/0238637 A1
(43) Pub. Date: Aug. 5, 2021

(54) METHOD FOR PRODUCING COENZYME Q10

(71) Applicant: KANEKA CORPORATION, Osaka-shi, Osaka (JP)

(72) Inventors: Yasuyuki SUZUKI, Takasago-shi (JP); Fumitaka KAMO, Takasago-shi (JP); Chanon KHUHAPREMA, Takasago-shi (JP); Akihisa KANDA, Takasago-shi (JP)

(73) Assignee: KANEKA CORPORATION, Osaka-shi, Osaka (JP)

(21) Appl. No.: 17/049,756

(22) PCT Filed: Apr. 25, 2019
(86) PCT No.: PCT/JP2019/017546

§ 371 Date: Oct. 22, 2020

(30) Foreign Application Priority Data
Apr. 27, 2018 (JP) 2018-087146

(51) Int. Cl. C12P 7/66; B01D 71/02; B01D 67/00; B01D 37/02

(52) U.S. Cl. CPC C12P 7/66; B01D 71/022; B01D 2201/202; B01D 37/02; B01D 67/0083

(57) ABSTRACT

Stable and economical methods for producing coenzyme Q10A are described. These methods include concentrating a culture suspension of a coenzyme Q10-producing microorganism efficiently, while minimizing a loss of coenzyme Q10 prior to an extraction step or a homogenization process. The methods for producing coenzyme Q10 may comprise a filtration step of passing a culture suspension of a coenzyme Q10-producing microorganism through a porous membrane in a state where the culture suspension is heated to a heating temperature of 35° C. or higher. In order to improve the average permeation flux, a regenerating step may be performed, preferably at least once, which involves closing a filtration device, applying pressure, and then releasing the pressure; or a regeneration treatment of allowing a medium to flow in from a filtrate outlet side and passing the medium through the porous membrane for a certain time, prior to normal filtration.

### TECHNICAL FIELD

The present invention relates to a method for producing coenzyme Q10. More specifically, the present invention relates to a method for producing coenzyme Q10 comprising a filtration step of passing a culture suspension of a coenzyme Q10-producing microorganism through a porous membrane.

### BACKGROUND ART

Coenzyme Q is an essential component, which is widely distributed in living organisms, from bacterium to mammals, and is known as a constituent of an electron transport system of mitochondria in living body cells. Coenzyme Q repeats oxidation and reduction to play a role as a messenger component in an electron transport system in a mitochondria. In addition, it is known that reduced coenzyme Q has an antioxidant property. Human coenzyme Q contains coenzyme Q10 having 10 repetitive structures in the side chain as a main component, and generally about 40 to 90% of human coenzyme Q exists in a reduced state in a living body. A physiological function of coenzyme Q is exemplified by an activation of an energy production by activating a mitochondria, an activation of cardiac function, a stabilization of a cell membrane, and a protection of cells by antioxidant action.

Many of the coenzyme Q10 that is currently manufactured and marketed is oxidized type, but recently reduced coenzyme Q10 having higher oral absorbability than oxidized coenzyme Q10 has been marketed and has been widely used.

Several methods for producing coenzyme Q10 have been known. For example, Patent document 1 discloses a method for producing reduced coenzyme Q10 including culturing a reduced coenzyme Q10-producing microorganism, optionally disrupting microbial cells, and extracting thus-produced reduced coenzyme Q10 by an organic solvent.

Patent document 2 discloses a method for producing coenzyme Q10 including contacting an extract of a coenzyme Q10-producing microorganism with an adsorbent singly, the adsorbent containing aluminum silicate as the main component, or with a plurality of adsorbents by combining the above adsorbent and another adsorbent. Patent document 2 describes that, according to the method of Patent document 2, a method for stably producing coenzyme Q10 with a simple coenzyme Q10 production step by efficiently removing impurity of a microorganism from an extract of coenzyme Q10-producing microorganism can be provided.

### PRIOR ART DOCUMENT

### Patent Document

[Patent document 1] JP 2008-253271 A

[Patent document 2] WO 2018/003974 A1

### SUMMARY OF THE INVENTION

### Problems to be Solved by the Invention

In the above-described conventional methods, there is still room for improvement for easy and mass production of coenzyme Q10 in a stable and effective manner.

For example, in the method of Patent document 1, there are problems that the concentration of culture suspended solids of the coenzyme Q10-producing microorganism is low, and the water content in the suspension is high, and thus upsizing of equipment for an extraction step is required, and in addition, it takes a great deal of time to disrupt the microbial cells, which are economically inefficient. Patent document 2 describes that the microorganism culture medium can be appropriately concentrated and then subjected to extraction, and in the examples, the microorganism culture medium is concentrated by centrifugation. However, when concentrated by a centrifuge, some coenzyme Q10-producing microorganism cells may flow out to a supernatant side, leading to a decrease in yield.

An objective of the present invention to solve the above-described problems is to provide a method for producing coenzyme Q10 including a filtration step performed prior to an extraction step, wherein the filtration step can concentrate a culture suspension of a coenzyme Q10-producing microorganism efficiently as much as possible while minimizing a loss of coenzyme Q10 in the culture suspension, and can be simply and stably operated.

### Solution to the Problems

The inventors of the present invention intensively studied for solving the above problems. As a result, the present inventors found that it is effective to provide, before an extraction step, a filtration step of passing a culture suspension of a coenzyme Q10-producing microorganism through a porous membrane under a specific temperature condition that the culture suspension of the coenzyme Q10-producing microorganism is heated to 35° C. or higher, and by the adoption of such a filtration step, after increasing the solid concentration of the culture suspension, the components in the microorganism can be extracted and then purified, whereby a loss of coenzyme Q10 can be minimized, and coenzyme Q10 can be efficiently purified. The present invention has been made based on such a finding.

The configuration of the method for producing coenzyme Q10 according to the present invention is as follows.

1. A method for producing coenzyme Q10 comprising, a filtration step of passing a culture suspension of a coenzyme Q10-producing microorganism through a porous membrane in a state where the culture suspension is heated to a heating temperature of 35° C. or higher.
2. The method according to the above 1, wherein, the heating temperature is 48° C. or higher.
3. The method according to the above 1 or 2, wherein, the culture suspension has a pH in a range of 3 to 7.
4. The method according to any one of the above 1 to 3, wherein, a linear velocity of the culture suspension in the filtration step is 0.1 m/s or more.
5. The method according to any one of the above 1 to 4, wherein, the filtration step comprises a regenerating step of closing a filtration device, applying pressure to the filtration device, and then releasing the pressure, and the regenerating step is performed at least once.
6. The method according to the above 5, wherein, the pressure applied to the filtration device is 0.1 to 1 MPa.
7. The method according to the above 5 or 6, wherein, a medium for applying pressure is at least one of air, nitrogen, water, the culture suspension, and a filtrate obtained by passing the culture suspension through the porous membrane.
8. The method according to any one of the above 1 to 7, further comprising, after a completion of the filtration step, a washing step of washing a microorganism suspended component adhered to the porous membrane, and then, performing the filtration step again.
9. The method according to the above 8, wherein, a wash liquid for the washing step is at least one of water, an aqueous alkaline solution, and an aqueous acidic solution.
10. The method according to the above 8 or 9, wherein, a temperature of the wash liquid is 10° C. to 90° C.
11. The method according to any one of the above 1 to 10, wherein, the porous membrane is made of any one of a synthetic resin, ceramic, and metal.
12. The method according to the above 11, wherein, the porous membrane is made of metal.
13. The method according to the above 12, wherein, the porous membrane made of metal is a separation layer of titanium oxide, and the separation layer is supported by a cylindrical support of stainless steel with an inner diameter of 5 mm or more.
14. The method according to the above 13, wherein, the separation layer has an average pore size of 0.01 to 3 gm.
15. The method according to any one of the above 1 to 14, further comprising, in the filtration step, a treatment step of allowing a medium to flow in from a filtrate outlet side and passing the medium through the porous membrane, and then performing the filtration step again.
16. The method according to the above 15, wherein, the medium for allowing to flow in is at least one of air, nitrogen, water, and a filtrate obtained by passing the culture suspension through the porous membrane.
17. The method according to the above 15 or 16, wherein, a temperature of the medium for allowing to flow in is 10° C. to 90° C.

### Advantageous Effects of the Invention

According to the present invention, it is possible to provide a method for producing coenzyme Q10 in which, by performing a filtration step of passing a culture suspension of a coenzyme Q10-producing microorganism through a porous membrane before an extraction step, the culture suspension of the coenzyme Q10-producing microorganism can be efficiently concentrated while minimizing a loss of coenzyme Q10 in the culture suspension. As a result, since a reduction in the use amount of a solvent when extracting or purifying coenzyme Q10, stabilization of extraction, and the like can be achieved, coenzyme Q10 can be efficiently produced also in terms of workability and economics.

### DESCRIPTION OF EMBODIMENTS

Hereinafter, one embodiment of the method for producing coenzyme Q10 according to the present invention is described, but the present invention is not restricted thereto.

The production method of the present invention is characterized by including a filtration step of passing a culture suspension of a coenzyme Q10-producing microorganism through a porous membrane in a state where the culture suspension is heated to 35° C. or higher. In the production method of the present invention, in the above filtration step using a porous membrane, coenzyme Q10 is extracted using an organic solvent from a microorganism cell suspension containing a coenzyme Q10-producing microorganism; or a concentrate obtained by concentrating a microorganism cell homogenate or an aqueous suspension of a microorganism cell homogenate, and the obtained coenzyme Q10 is brought into contact with an aqueous alkaline solution or water as needed, whereby coenzyme Q10 that is purified or has improved purity can be separated and recovered.

Hereinafter, the production method of the present invention will be described in detail.

(1) Coenzyme Q10-Producing Microorganism Used in the Present Invention

Coenzyme Q10 includes an oxidized type and a reduced type. The target of the present invention is both of oxidized coenzyme Q10 and reduced coenzyme Q10 as coenzyme Q10, and coenzyme Q10 containing both of oxidized coenzyme Q10 and reduced coenzyme Q10 is also the target of the present invention. When coenzyme Q10 is a mixture of oxidized coenzyme Q10 and reduced coenzyme Q10, a content ratio of reduced coenzyme Q10 is not particularly restricted. The description of mere “coenzyme Q10” in this disclosure represents any of oxidized coenzyme Q10, reduced coenzyme Q10, and a mixture of oxidized coenzyme Q10 and reduced coenzyme Q10.

As the coenzyme Q10-producing microorganism usable in the present invention, any one of bacteria, yeast and fungus may be used without limitation as long as the microorganism can produce coenzyme Q10 in the microorganism. Such a microorganism is specifically exemplified by a microorganism belonging to genera of Acetobacter, Aminobacter, Agromonas, Acidiphilium, Bulleromyces, Bullera, Brevundimonas, Cryptococcus, Chionosphaera, Candida, Cerinosterus, Exisophiala, Exobasidium, Fellomyces, Filobasidiella, Filobasidium, Geotrichum, Graphiola, Gluconobacter, Kockovaella, Kurtzmanomyces, Lalaria, Leucosporidium, Legionella, Methylobacterium, Mycoplana, Oosporidium, Pseudomonas, Psedozyma, Paracoccus, Petromyc, Rhodotorula, Rhodosporidium, Rhizomonas, Rhodobium, Rhodoplanes, Rhodopseudomonas, Rhodobacter, Sporobolomyces, Sporidiobolus, Saitoella, Schizosaccharomyces, Sphingomonas, Sporotrichum, Sympodiomycopsis, Sterigmatosporidium, Tapharina, Tremella, Trichosporon, Tilletiaria, Tilletia, Tolyposporium, Tilletiopsis, Ustilago, Udeniomyce, Xanthophllomyces, Xanthobacter, Paecilomyces, Acremonium, Hyhomonus, Rhizobium, Phaffia and Haematococcus.

Among them, from the aspect of easy cultivation and productivity, bacteria and yeast are preferred. As bacteria, non-photosynthetic bacteria is preferred, and further, bacteria belonging to genera of Agrobacterium and Gluconobacter are particularly preferred. As yeast, a yeast belonging to genera of Schizosaccharomyces, Saitoella and Phaffia are particularly preferred.

When reduced coenzyme Q10 is purposely produced as coenzyme Q10, it is preferred to use a microorganism by which produced coenzyme Q10 has high content ratio of reduced coenzyme Q10. For example, it is more preferred to use a microorganism of which content ratio by weight of reduced coenzyme Q10 in coenzyme Q10 after cultivation is preferably 70% or more and more preferably 80% or more.

As the coenzyme Q10-producing microorganism used in the present invention, not only a wild strain of the above-described microorganism but also a variant and a recombinant of the above-described microorganism of which transcription activity and translation activity of a gene involved in a biosynthesis of the target coenzyme Q10 and an enzyme activity of an expressed protein is altered or improved can be used.

By cultivating the above-described microorganism, microorganism cells containing coenzyme Q10 can be obtained. A cultivating method is not particularly restricted, and a cultivating method suitable for the target microorganism or the production of the target coenzyme Q10 can be appropriately selected. A cultivation time is also particularly not restricted, and may be adjusted to the range that a desired amount of the target coenzyme Q10 is accumulated in microorganism cells.

In the present invention, it is preferred that the culture suspension of the coenzyme Q10-producing microorganism obtained by the above method be passed through the porous membrane. However, the present invention is not limited to this, and a culture suspension of a coenzyme Q10-producing microorganism obtained by another method may be passed through the porous membrane. For example, microorganism cells are once subjected to solid-liquid separation or dried with a filter press, then re-suspended in water to obtain a suspension (suspension of dried microorganism cell homogenate), and the obtained suspension may be used. Furthermore, as described later, a microorganism cell homogenate or an aqueous suspension of a microorganism cell homogenate may be used. The concentration of microorganism in the culture suspension to be passed through the porous membrane is not particularly limited, and is preferably in the range of 0.01 to 10 wt % in terms of dry weight of the microorganism.

(2) Filtration Step

In the production method of the present invention, the above-described culture suspension needs to be heated to 35° C. or higher and then passed through the porous membrane. If the heating temperature is lower than 35° C., the filtration effect may be reduced due to the growth of unwanted bacteria in the culture suspension. The heating temperature is not particularly limited as long as it is 35° C. or higher. To further improve the filtration effect, the heating temperature is preferably 40° C. or higher, and more preferably 48° C. or higher. The upper limit thereof is not limited, and the heating temperature is preferably 90° C. or lower, and more preferably 60° C. or lower from the viewpoints of operability and quality. In the filtration step, the heating temperature does not have to be constant and may be changed within the above range during the filtration operation.

The pH of the culture suspension when being passed through the porous membrane is preferably in the range of 3 to 7, more preferably in the range of 3 to 5, and still more preferably in the range of 3.5 to 4.5. If the pH of the obtained culture suspension satisfies these ranges, the culture suspension can be used as it is, and if not, acid or alkali can be used to adjust the pH of the culture suspension to a desired pH. By adjusting the pH of the culture suspension to be within these ranges, the precipitation of inorganic salts in the culture suspension is suppressed, thereby not only preventing clogging in the porous membrane but also reducing the viscosity of the culture suspension, and thus the speeding-up of the filtration step with the porous membrane can be achieved.

In the above filtration step, the linear velocity when the microorganism culture suspension is passed through the porous membrane is not particularly limited, and is preferably 0.1 m/s or more, more preferably 1 m/s or more, and further preferably 3 m/s or more. The faster linear velocity makes it possible to speed up the filtration step.

In the above filtration step, the permeation flux when the microorganism culture suspension is passed through the porous membrane is not particularly limited unless the porous membrane gets completely clogged, and is preferably higher in view of economics such as equipment cost and production amount. The average permeation flux throughout the filtration step is preferably 0.50 kg/min/m² or more, and more preferably 1.0 kg/min/m² or more.

The type of the porous membrane through which the culture suspension of the coenzyme Q10-producing microorganism is passed is not particularly limited. Preferable examples thereof include a porous membrane made of a synthetic resin, ceramic, or metal.

The above synthetic resin is not particularly limited as long as it has a molecular weight capable of withstanding a fluid passage. Examples of the synthetic resin include polyethylene, polypropylene, polymethylmethacrylate, polystyrene, and fluororesin. In the light of prices and availability, polyethylene, polypropylene, and polystyrene are preferable.

Examples of the above ceramic include oxide ceramics such as alumina, zirconia, and barium titanate, hydroxide ceramics such as hydroxyapatite, carbides such as silicon carbide, nitride ceramics such as silicon nitride, halide ceramics such as fluorite, and phosphate ceramics. In the light of versatility and availability, oxide ceramics are preferable, and alumina is more preferable.

Examples of the above metal include iron, copper, zinc, tin, mercury, lead, aluminum, titanium, titanium oxide, and stainless steel. In the light of acid resistance, alkali resistance, and strength, stainless steel or titanium oxide is preferable.

In the production method of the present invention, it is preferable to use a metal porous membrane because a high regeneration effect can be obtained during a regeneration step. More preferably, the above porous membrane includes a separation layer made of titanium oxide, which corresponds to a filter, and an external body (support) made of stainless steel that supports the separation layer. In the present invention, the “separation layer (filter)” is not particularly limited as long as it is not permeable to microorganism cells in the culture suspension or a microorganism cell homogenate but permeable to a water-soluble medium portion. The separation layer may be in any form of mesh, non-woven fabric, and fine-pored.

In the production method of the present invention, the shape of the porous membrane for filtering the culture suspension of the coenzyme Q10-producing microorganism is not particularly limited, and is preferably cylindrical in terms of operation and equipment installation. In particular, when the porous membrane is composed of the separation layer (filter) and the external body (support) that supports the separation layer as described above, the porous membrane is more preferably a cylinder including a hollow body having the separation layer made of titanium oxide or the like and a cylindrical support made of stainless steel or the like in which the hollow body is housed. Specifically, for example, by configuring the porous membrane including the hollow porous body (titanium oxide) housed in the external body (stainless steel), a concentrated slurry passes through the hollow portion, a filtrate is discharged to the outside, and thus filtration and concentration proceed. The cylinder used in the present invention is desirably lightweight and has a small diameter in view of easy handling. In view of the solid concentration and the viscosity of the culture suspension, the cylinder has an inner diameter of preferably 2 mm or more, and more preferably 5 mm or more. The upper limit of the inner diameter is not particularly limited from the above viewpoints, but is preferably about 30 mm in consideration of weight of equipment and securing of a specific surface area.

The average pore size of the separation layer is preferably in the range of 0.01 to 3 gm considering the particle sizes of the microorganism in the culture suspension and other solids produced by culturing the microorganism. Further considering productivity, strength, difficulty in clogging, and easiness of regeneration, the average pore size of the separation layer is more preferably 0.05 gm or more, and is preferably 1 gm or less, and more preferably 0.8 gm or less. The separation layer of the porous membrane having an average pore size within the above range can attain a high yield without leaking solids containing coenzyme Q10 in the filtrate.

In the production method of the present invention, the microorganism cell suspension can be concentrated by performing the above filtration step. The concentration of microorganism in the concentrated microorganism cell suspension after the filtration step is not particularly limited. The concentration step is preferably performed such that the concentration of the microorganism becomes 0.1 to 25 wt % in terms of dry weight of the microorganism. Considering stability and economics, the concentration step is more preferably performed such that the concentration of the microorganism becomes within the range of 10 to 20 wt %.

In the filtration step, in order to improve the average permeation flux, the temperature may be changed during the operation, or a regeneration operation may be appropriately introduced.

When the passing the microorganism culture suspension through the porous membrane is continued, solids derived from the microorganism culture suspension are adhered to the inside and the surface of the porous membrane, and the filtration rate and the permeation flux tend to gradually decrease. Therefore, in the production method of the present invention, a regenerating step of closing a filtration device, applying pressure to the filtration device, and then releasing the pressure is preferably performed at least once during the passing of the culture suspension through the porous membrane. By performing this regeneration treatment, the solids adhered to the inside and the surface of the porous membrane can be removed to the outside of the filtration device, and the filtration speed can be recovered, resulting in speeding-up of the filtration step.

The above-described regeneration step is performed temporarily during the passing of the culture suspension through the porous membrane (that is, in at least a partial time period during the passing process of the culture suspension), and it is not intended to perform the regeneration treatment throughout the passing process of the culture suspension. Specifically, for example, an operation in which an outlet is temporarily closed to apply pressure while the culture suspension fed from an inlet is continuously passed through the porous membrane, and then the outlet is opened to restore the pressure in the system is conducted during the passing of the culture suspension through the porous membrane, whereby the regeneration step of applying and releasing pressure can be accomplished. Alternatively, an operation in which either an inlet or outlet is temporarily closed, pressure is applied by injecting a medium into the system from the outlet or inlet on the opposite side, and then the closed inlet or outlet is opened to restore the pressure in the system is conducted during the passing of the culture suspension through the porous membrane, whereby the regeneration step of applying and releasing pressure can be accomplished. The conduct time period of such a regeneration step is, for example, 10% or less, preferably 5% or less, and more preferably 2% or less of the entire time period of the passing process.

A method of applying pressure is not particularly limited. A medium for applying pressure is at least one of air, nitrogen, water, a microorganism cell suspension, and a filtrate obtained by passing the microorganism cell suspension through the filtration membrane (hereinafter, may be simply referred to as a filtrate). The more preferred medium is air.

The pressure when the filtration device is closed to apply pressure is not particularly limited, and is preferably in the range of 0.1 to 1 MPa. Considering the washing effect, regeneration effect, and device safety, the pressure is more preferably 0.2 MPa or more, and is more preferably 0.6 MPa or less.

A time period until the pressure is released after applying the pressure is not particularly limited, and from the viewpoint of productivity, it is desirable to perform the regeneration treatment in a time period as short as possible, preferably within 5 minutes, more preferably within 1 minute, and further preferably within 20 seconds.

The regeneration treatment may be performed at least once during the passing of the culture suspension. If the regeneration treatment is performed multiple times, there are problems that the treatment time becomes long and the operation becomes complicated. Therefore, the upper limit of the number of regeneration treatments is, for example, 100 times, and preferably 60 times.

In the production method of the present invention, after the completion of the filtration step, it is preferred to perform a washing step of washing a microorganism suspended component adhered to the porous membrane, and then the filtration step again. This allows the porous membrane to be used for the long term. A wash liquid for washing the microorganism suspended component adhered to the porous membrane is not particularly limited, and at least one of water, an aqueous alkaline solution and an aqueous acidic solution can be used as the wash liquid. Specifically, the type of wash liquid can be appropriately selected according to the type of object to be washed. For example, it is recommended to use an aqueous alkaline solution for washing an organic substance and an aqueous acidic solution for washing an inorganic substance. An aqueous alkaline solution and an aqueous acidic solution are preferably used, and it is more preferable to perform both washing with the aqueous alkaline solution and washing with the aqueous acidic solution.

The type of the aqueous alkaline solution is not particularly limited, and examples thereof include aqueous ammonia, sodium hydroxide, potassium hydroxide, lithium hydroxide, sodium carbonate, sodium hydrogen carbonate, magnesium oxide, calcium hydroxide, and sodium acetate. An aqueous solution of sodium hydroxide or potassium hydroxide is preferable in consideration of economics. The concentration of the aqueous alkaline solution is also not particularly limited, and is preferably 5 wt % or less in consideration of handleability.

The type of the aqueous acidic solution is not particularly limited, and examples thereof include hydrochloric acid, nitric acid, sulfuric acid, phosphoric acid, acetic acid, carbonic acid, oxalic acid, sulfamic acid, citric acid, and hydrogen sulfide. An aqueous solution of sulfuric acid, sulfamic acid, or citric acid is preferable in consideration of economics and handleability. The concentration of the aqueous acidic solution is also not particularly limited, and is preferably 5 wt % or less in consideration of economics and handleability.

The temperature of the wash liquid in the washing step is preferably a high temperature, and is preferably 10° C. or higher, more preferably 40° C. or higher, and further preferably 65° C. or higher. Washing at a higher temperature allows the regeneration efficiency of the porous membrane to be improved. The upper limit of the temperature is not particularly limited, and is preferably 90° C. or lower.

In the production method of the present invention, in addition to the above-described regeneration method, as a regeneration method of the porous membrane, during the passing the culture suspension, it may be possible to perform a regeneration treatment step of allowing a medium to flow in from a filtrate outlet side and passing the medium through the porous membrane, and then the filtration step again. The medium for allowing to flow in is not particularly limited, and air, nitrogen, water, a filtrate, an aqueous alkaline solution, an aqueous acidic solution or the like can be used. From the viewpoints of economics, safety and quality, it is preferable to use air, nitrogen, water or a filtrate.

The above-described regeneration step is performed temporarily during the passing the culture suspension (that is, in at least a partial time period during the passing process), and it is not intended to perform the regeneration treatment throughout the passing process of the culture suspension. Specifically, for example, a method can be given in which the medium is allowed to flow in from the filtrate outlet side and passed in the reverse direction for a predetermined time period, and then the passing of the culture suspension is restarted from the inlet side. As for the timing of the above regeneration treatment, which varies depending on the allowable treatment time and the like, for example, the regeneration treatment may be performed when the average permeation flux decreases (roughly to 0.2 L/min/m² or less); or the regeneration treatment may be performed, for example, every hour regardless of the average permeation flux.

Furthermore, in the above-described regeneration step, the time period during which the medium is allowed to flow in and passed through is not particularly limited, and may be a time period required to increase a decreased filtration rate to a predetermined value. For example, it is recommended that one regeneration treatment is normally performed for 5 seconds or more, preferably 15 seconds or more, and more preferably 30 seconds or more.

The temperature of the medium for allowing to flow in to regenerate the porous membrane is not particularly limited, and is preferably 10° C. to 90° C. Considering that the temperature of the filtration step is prevented from largely changing and the washing recovery performance is ensured, the temperature of the medium is more preferably 30° C. or higher, and is more preferably 70° C. or lower.

(3) Homogenization as Needed

In the production method of the present invention, coenzyme Q10 is extracted using an organic solvent after the filtration step. In the extraction of coenzyme Q10, coenzyme Q10 can be directly extracted from the microorganism cell culture concentrate obtained through the filtration step of passing through the porous membrane as described above, or, preferably, the microorganism cells in the concentrate are homogenized to obtain a microorganism cell homogenate or an aqueous suspension of a microorganism cell homogenate, and then coenzyme Q10 is extracted from the microorganism cell homogenate or the aqueous suspension of the microorganism cell homogenate. Alternatively, the microorganism cells are dried, and coenzyme Q10 can be extracted from the dried microorganism cell.

In the present invention, homogenization of the microorganism cell culture medium may be performed first, and then concentration may be performed by passing the culture suspension or aqueous suspension of the obtained microorganism cell homogenate through the porous membrane. Namely, the order of the filtration step (2) and the homogenization step (3) does not matter. However, it is preferable to perform the filtration step (2) prior to the homogenization step (3).

In the “homogenization” in the present invention, the surface structure of a cell wall or the like is damaged so that it becomes possible to extract the target coenzyme Q10.

The homogenization method is exemplified by physical treatment and chemical treatment.

The above-described physical treatment is exemplified by a treatment using high pressure homogenizer, rotary blade homogenizer, ultrasonic homogenizer, French press, ball mill or the like, and a combination thereof.

The above-described chemical treatment is exemplified by a treatment using an acid such as hydrochloric acid and sulfuric acid, preferably a strong acid, a base such as sodium hydroxide and potassium hydroxide, preferably a strong base, and a combination thereof.

As a method for homogenizing cells as a pretreatment for the extraction and recovery of coenzyme Q10 in the present invention, a physical treatment is more preferred among the above-described homogenization methods in terms of a homogenization efficiency

In the production method of the present invention, as described above, the culture suspension containing the coenzyme Q10-producing microorganism obtained as described above can be concentrated by the filtration step and then dried, and coenzyme Q10 can be extracted from the dried microorganism cells. A dryer for drying the microorganism cells in this case is exemplified by fluidized dryer, spray dryer, box dryer, cone dryer, cylindrical vibration dryer, cylindrical agitating dryer, inverse cone dryer, filter dryer, freeze dryer, microwave dryer, and a combination thereof.

A water concentration in the dried microorganism cells is preferably included in the range of 0 to 50 wt%. In addition, a dried microorganism homogenate prepared by further homogenizing the dried microorganism cells with the above-described homogenization method or drying the above-described microorganism cell homogenate can be also used.

(4) Extraction Step

In the production method of the present invention, an organic solvent usable for the extraction of coenzyme Q10 is not particularly restricted and exemplified by a hydrocarbon solvent, a fatty acid ester solvent, an ether solvent, an alcohol solvent, a fatty acid solvent, a ketone solvent, a nitrogen compound solvent such as a nitrile solvent and an amide solvent, and a sulfur compound solvent.

The hydrocarbon solvent is not particularly restricted and is exemplified by an aliphatic hydrocarbon solvent, an aromatic hydrocarbon solvent and a halogenated hydrocarbon solvent. Among the examples, an aliphatic hydrocarbon solvent and an aromatic hydrocarbon solvent are preferred, and an aliphatic hydrocarbon solvent is more preferred.

An aliphatic hydrocarbon solvent may be cyclic or non-cyclic and saturated or unsaturated, is not particularly restricted, and a saturated aliphatic hydrocarbon solvent is generally used. In general, a C₃₋₂₀ aliphatic hydrocarbon solvent is used, a C₅₋₁₂ aliphatic hydrocarbon solvent is preferably used, and a C₅₋₈ aliphatic hydrocarbon solvent is more preferably used. Specifically, propane, butane, isobutane, pentane, 2-methylbutane, hexane, 2-methylpentane, 2,2-dimethylbutane, 2,3-dimethylbutane, heptane, a heptane isomer such as 2-methylhexane, 3-methylhexane, 2,3-dimethylpentane and 2,4-dimethylpentane, octane, 2,2,3-trimethylpentane, isooctane, nonane, 2,2,5-trimethylhexane, decane, dodecane, 2-pentene, 1-hexene, 1-heptene, 1-octene, 1-nonene, 1-decene, cyclopentane, methylcyclopentane, cyclohexane, methylcyclohexane, ethylcyclohexane, p-menthane and cyclohexene are exemplified. Preferably, pentane, 2-methylbutane, hexane, 2-methylpentane, 2,2-dimethylbutane, 2,3-dimethylbutane, heptane, 2-methylhexane, 3-methylhexane, 2,3-dimethylpentane, 2,4-dimethylpentane, octane, 2,2,3-trimethylpentane, isooctane, nonane, 2,2,5-trimethylhexane, decane, dodecane, cyclopentane, methylcyclopentane, cyclohexane, methylcyclohexane, ethylcyclohexane and p-menthane are exemplified. More preferably, pentane, 2-methylbutane, hexane, 2-methylpentane, 2,2 -dimethylbutane, 2,3-dimethylbutane, heptane, 2-methylhexane, 3-methylhexane, 2,3-dimethylentane, 2,4-dimethylpentane, octane, 2,2,3-trimethylpentane, isooctane, cyclopentane, methylcyclopentane, cyclohexane, methylcyclohexane and ethylcyclohexane are exemplified. More preferably, pentane, hexane, cyclohexane and methylcyclohexane are exemplified. Particularly preferably, heptane, hexane and methylcyclohexane are exemplified, in view of very high protection effect from oxidation and versatility. Most preferably, heptane and hexane are exemplified.

An aromatic hydrocarbon solvent is not particularly restricted, and a C₆₋₂₀ aromatic hydrocarbon solvent is generally used, a C₆₋₁₂ aromatic hydrocarbon solvent is preferably used, and a C₇₋₁₀ aromatic hydrocarbon solvent is more preferably used. Specifically, an aromatic hydrocarbon solvent is exemplified by benzene, toluene, xylene, o-xylene, m-xylene, p-xylene, ethylbenzene, cumene, mesitylene, tetralin, butylbenzene, p-cymene, cyclohexylbenzene, diethylbenzene, pentylbenzene, dipentylbenzene, dodecylbenzene and styrene, preferably toluene, xylene, o-xylene, m-xylene, p-xylene, ethylbenzene, cumene, mesitylene, tetralin, butylbenzene, p-cymene, cyclohexylbenzene, diethylbenzene and pentylbenzene, more preferably toluene, xylene, o-xylene, m-xylene, p-xylene, cumene and tetralin, and most preferably cumene.

A halogenated hydrocarbon solvent may be cyclic or non-cyclic and saturated or unsaturated, is not particularly restricted, and a non-cyclic halogenated hydrocarbon solvent is preferably used. A halogenated hydrocarbon solvent is more preferably a chlorinated hydrocarbon and a fluorinated hydrocarbon, and even more preferably a chlorinated hydrocarbon.

In addition, a C₁₋₆ halogenated hydrocarbon solvent may be used, a C₁₋₄ halogenated hydrocarbon solvent is preferably used, and a C₁₋₂ halogenated hydrocarbon solvent is more preferably used. Specifically, a halogenated hydrocarbon solvent is exemplified by dichloromethane, chloroform, carbon tetrachloride, 1,1-dichloroethane, 1,2-dichloroethane, 1,1,1-trichloroethane, 1,1,2-trichloroethane, 1,1,1,2-tetrachloroethane, 1,1,2,2-tetrachloroethane, pentachloroethane, hexachloroethane, 1,1- dichloroethylene, 1,2-dichloroethylene, trichloroethylene, tetrachloroethylene, 1,2-dichloropropane, 1,2,3-trichlorop rop ane, chlorobenzene and 1,1,1,2-tetrafluoroethane, preferably dichloromethane, chloroform, carbon tetrachloride, 1,1-dichloroethane, 1,2-dichloroethane, 1,1,1-trichloroethane, 1,1,2-trichloroethane, 1,1-dichloroethylene, 1,2-dichloroethylene, trichloroethylene, chlorobenzene and 1,1,1,2-tetrafluoroethane, more preferably dichloromethane, chloroform, 1,2-dichloroethylene, trichloroethylene, chlorobenzene and 1,1,1,2-tetrafluoroethane.

A fatty acid ester solvent is not particularly restricted, and is exemplified by a propionate ester, an acetate ester and a formate ester, preferably an acetate ester and a formate ester, and more preferably an acetate ester. An ester group is not particularly restricted, and a C₁₋₈ alkyl ester and a C₇₋₁₂ aralkyl ester are generally used, a C₁₋₆ alkyl ester is preferably used, and a C₁₋₄ alkyl ester is more preferably used.

A propionate ester is specifically exemplified by methyl propionate, ethyl propionate, butyl propionate and isopentyl propionate, and preferably ethyl propionate.

An acetate ester is specifically exemplified by methyl acetate, ethyl acetate, propyl acetate, isopropyl acetate, butyl acetate, isobutyl acetate, sec-butyl acetate, pentyl acetate, isopentyl acetate, sec-hexyl acetate, cyclohexyl acetate and benzyl acetate, preferably methyl acetate, ethyl acetate, propyl acetate, isopropyl acetate, butyl acetate, isobutyl acetate, sec-butyl acetate, pentyl acetate, isopentyl acetate, sec-hexyl acetate and cyclohexyl acetate, more preferably methyl acetate, ethyl acetate, propyl acetate, isopropyl acetate, butyl acetate and isobutyl acetate, and most preferably ethyl acetate.

A formate ester is specifically exemplified by methyl formate, ethyl formate, propyl formate, isopropyl formate, butyl formate, isobutyl formate, sec-butyl formate and pentyl formate, preferably methyl formate, ethyl formate, propyl formate, butyl formate, isobutyl formate and pentyl formate, and most preferably ethyl formate.

An ether solvent may be cyclic or non-cyclic and saturated or unsaturated, is not particularly restricted, and a saturated ether solvent is preferably used. In general, a C₃₋₂₀ ether solvent is used, a C₄₋₁₂ ether solvent is preferably used, and a C₄₋₈ ether solvent is more preferably used. An ether solvent is specifically exemplified by diethyl ether, methyl tert-butyl ether, dipropyl ether, diisopropyl ether, dibutyl ether, dihexyl ether, ethyl vinyl ether, butyl vinyl ether, anisole, phenetol, butyl phenyl ether, methoxytoluene, dioxane, furan, 2-methylfuran, tetrahydrofuran, tetrahydropyran, ethylene glycol dimethyl ether, ethylene glycol diethyl ether, ethylene glycol dibutyl ether, ethylene glycol monomethyl ether, ethylene glycol monoethyl ether and ethylene glycol monobutyl ether, preferably diethyl ether, methyl tert-butyl ether, dip ropyl ether, diisopropyl ether, dibutyl ether, dihexyl ether, anisole, phenetol, butyl phenyl ether, methoxytoluene, dioxane, 2-methylfuran, tetrahydrofuran, tetrahydropyran, ethylene glycol dimethyl ether, ethylene glycol diethyl ether, ethylene glycol dibutyl ether, ethylene glycol monomethyl ether and ethylene glycol monoethyl ether, more preferably diethyl ether, methyl tert-butyl ether, anisole, dioxane, tetrahydrofuran, ethylene glycol monomethyl ether and ethylene glycol monoethyl ether, more preferably diethyl ether, methyl tert-butyl ether and anisole, and most preferably methyl tert-butyl ether.

An alcohol solvent may be cyclic or non-cyclic and saturated or unsaturated, is not particularly restricted, and a saturated alcohol solvent is generally used. In general, a C₁₋₂₀ alcohol solvent is used, a C₁₋₁₂ alcohol solvent is preferably used, and a C₁₋₆ alcohol solvent is more preferably used. In particular, a C₁₋₅ monovalent alcohol solvent, a C₂₋₅ divalent alcohol solvent and a C₃ trivalent alcohol solvent are preferred.

An alcohol solvent is specifically exemplified by a monovalent alcohol such as methanol, ethanol, 1-propanol, 2-propanol, 1-butanol, 2 -butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, neopentyl alcohol, 1-hexanol, 2-methyl-1-pentanol, 4-methyl-2-pentanol, 2-ethyl-1-butanol, 1-heptanol, 2-heptanol, 3-heptanol, 1-octanol, 2-octanol, 2-ethyl-l-hexanol, 1-nonanol, 1-decanol, 1-undecanol, 1-dodecanol, allyl alcohol, propargyl alcohol, benzyl alcohol, cyclohexanol, 1-methylcyclohexanol, 2-methylcyclohexanol, 3-methylcyclohexanol and 4-methylcyclohexanol; a divalent alcohol solvent such as 1,2-ethanediol, 1,2-propanediol, 1,3-propanediol, 1,2-butanediol, 1,3-butanediol, 1,4-butanediol, 2,3-butanediol and 1,5-pentanediol; and a trivalent alcohol such as glycerin.

A monovalent alcohol is preferably methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, neopentyl alcohol, 1-hexanol, 2-methyl-1-pentanol, 4-methyl-2-pentanol, 2-ethyl-1-butanol, 1-heptanol, 2 -heptanol, 3-heptanol, 1-octanol, 2-octanol, 2-ethyl-1-hexanol, 1-nonanol, 1-decanol, 1-undecanol, 1-dodecanol, benzyl alcohol, cyclohexanol, 1- methylcyclohexanol, 2- methylcyclohexanol, 3- methylcyclohexanol or 4-methylcyclohexanol, more preferably methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol, neopentyl alcohol, 1-hexanol, 2-methyl-1-pentanol, 4-methyl-2-pentanol, 2-ethyl-1-butanol or cyclohexanol, more preferably methanol, ethanol, 1-propanol, 2-propanol, 1-butanol, 2-butanol, isobutyl alcohol, tert-butyl alcohol, 1-pentanol, 2-pentanol, 3-pentanol, 2-methyl-1-butanol, isopentyl alcohol, tert-pentyl alcohol, 3-methyl-2-butanol or neopentyl alcohol, particularly preferably methanol, ethanol, 1-propanol, 2-propanol, 1-butanol, 2-butanol, isobutyl alcohol, 2-methyl-1-butanol or isopentyl alcohol, and most preferably 2-propanol.

A divalent alcohol is preferably 1,2-ethanediol, 1,2-propanediol or 1,3-propanediol, and most preferably 1,2-ethanediol. A trivalent alcohol is preferably glycerin.

A fatty acid solvent is exemplified by formic acid, acetic acid and propionic acid, preferably formic acid and acetic acid, and most preferably acetic acid.

A ketone solvent is not particularly restricted, and a C₃₋₆ ketone solvent is preferably used. A ketone solvent is specifically exemplified by acetone, methyl ethyl ketone, methyl butyl ketone and methyl isobutyl ketone, preferably acetone and methyl ethyl ketone, and most preferably acetone.

A nitrile solvent may be cyclic or non-cyclic and saturated or unsaturated, is not particularly restricted, and a saturated nitrile solvent is generally used. In general, a C₂₋₂₀ nitrile solvent is used, a C₂₋₁₂ nitrile solvent is preferably used, and a C₂₋₈ nitrile solvent is more preferably used.

A nitrile solvent is specifically exemplified by acetonitrile, propionitrile, malononitrile, butyronitrile, isobutyronitrile, succinonitrile, valeronitrile, glutaronitrile, hexanenitrile, heptyl cyanide, octyl cyanide, undecanenitrile, dodecanenitrile, tridecanenitrile, pentadecanenitrile, stearonitrile, chloroacetonitrile, bromoacetonitrile, chlorop rop ionitrile, bromop rop ionitrile, methoxyacetonitrile, methyl cyanoacetate, ethyl cyanoacetate, tolunitrile, benzonitrile, chlorobenzonitrile, bromobenzonitrile, cyanobenzoic acid, nitrobenzonitrile, anisonitrile, phthalonitrile, bromotolunitrile, methyl cyanobenzoate, methoxybenzonitrile, acetylbenzonitrile, nap hthonitrile, b ip henylcarbonitrile, p henylp rop ionitrile, p henylbutyronitrile, methylp henylacetonitrile, dip henylacetonitrile, nap hthylacetonitrile, nitrop henylacetonitrile, chlorobenzyl cyanide, cyclop rop anecarbonitrile, cyclohexanecarb onitrile, cycloheptanecarbonitrile, phenylcyclohexanecarbonitrile and tolylcyclohexanecarbonitrile.

A nitrile solvent is preferably acetonitrile, propionitrile, succinonitrile, butyronitrile, isobutyronitrile, valeronitrile, methyl cyanoacetate, ethyl cyanoacetate, benzonitrile, tolunitrile or chloropropionitrile, more preferably acetonitrile, propionitrile, butyronitrile or isobutyronitrile, and most preferably acetonitrile.

A nitrogen compound solvent except for a nitrile solvent is exemplified by an amide solvent such as formamide, N-methylformamide, N,N-dimethylformamide, N, N-dimethylacetamide and N-methylpyrrolidone; nitromethane; triethylamine; and pyridine.

A sulfur compound solvent is exemplified by dimethyl sulfoxide and sulfolane.

It is preferred to select an organic solvent used in the present invention in consideration of the boiling point, melting point, viscosity, or the like. For example, as for the boiling point, an organic solvent having a boiling point in the range of about 30 to 150° C. at 1 atmosphere is preferably used, since such an organic solvent can be moderately heated for increasing a solubility and easily removed and exchanged. As for the melting point, an organic solvent having a melting point of about 0° C. or higher, preferably about 10° C. or higher, and more preferably about 20° C. or higher may be used, since such an organic solvent is hardly solidified when using the solvent at room temperature and even when cooling the solvent to room temperature or lower. As for the viscosity, an organic solvent having a low viscosity of about 10 cp or lower at 20° C. is preferably used.

In the production method of the present invention, among the above organic solvents, a hydrophobic organic solvent or an organic solvent containing a hydrophobic organic solvent is preferably used as an extraction solvent when coenzyme Q10 is extracted from an aqueous concentrated suspension of microorganism cells or a microorganism cell homogenate. Use of an organic solvent prepared by mixing a hydrophobic organic solvent with a small amount of a hydrophilic organic solvent (for example, an alcohol solvent such as isopropanol) or with a surfactant can further enhance the extraction efficiency of coenzyme Q10.

The hydrophobic organic solvent used in this case is not particularly restricted, and a hydrophobic solvent among the above-described organic solvents may be used. A hydrocarbon solvent, a fatty acid ester solvent and an ether solvent are preferably used, a fatty acid ester solvent and a hydrocarbon solvent are more preferably used, and an aliphatic hydrocarbon solvent is further preferably used.

As the aliphatic hydrocarbon solvent, a C₅₋₈ aliphatic hydrocarbon solvent is preferably used. The C₅₋₈ aliphatic hydrocarbon solvent is specifically exemplified by pentane, 2-methylbutane, hexane, 2-methylpentane, 2,2-dimethylbutane, 2,3-dimethylbutane, heptane, 2-methylhexane, 3-methylhexane, 2,3-dimethylpentane, 2,4-dimethylpentane, octane, 2,2,3-trimethylpentane, isooctane, cyclopentane, methylcyclopentane, cyclohexane, methylcyclohexane and ethylcyclohexane, particularly preferably hexane, heptane and methylcyclohexane, and most preferably hexane.

Further, as the aliphatic acid ester solvent, ethyl acetate is preferably used.

An amount of the extraction solvent used in the production method of the present invention is not particularly restricted, and the concentration of the extraction solvent at the time of the extraction is preferably in the range of 25 to 80 vol %, and more preferably in the range of 50 to 75 vol % to the entire solution volume. A temperature at the time of the extraction in the production method of the present invention is not particularly restricted and may be generally in the range of 0 to 60° C. and preferably in the range of 20 to 50° C.

An extraction method may be any one of batch extraction and continuous extraction, and continuous extraction is industrially preferred in terms of a productivity. Among continuous extraction, countercurrent multistep extraction is particularly preferred. A stirring time in the case of batch extraction is not particularly restricted and may be generally 5 minutes or longer. An average residence time in the case of continuous extraction is not particularly restricted and may be generally 10 minutes or longer.

(5) Separation and Removal of Solid

In the production method of the present invention, the extract of the coenzyme Q10-producing microorganism obtained as described above may be directly cooled to precipitate a solid, or may be brought into contact with an aqueous alkaline solution to saponify a fat-soluble component derived from the microorganism, washed with water, and then concentrated to obtain a concentrated extract, and the concentrated extract may be cooled to precipitate a solid. By thus separating and removing the solid, impurities in the extract are removed, so that the extract with high purity coenzyme Q10 can be obtained.

Examples of the aqueous alkaline solution for saponification of the extract of the coenzyme Q10-producing microorganism include aqueous ammonia, an aqueous sodium hydroxide solution, an aqueous potassium hydroxide solution, an aqueous lithium hydroxide solution, an aqueous sodium carbonate solution, an aqueous sodium hydrogen carbonate solution, a magnesium oxide aqueous solution, an aqueous calcium hydroxide solution, an aqueous sodium acetate solution, and the like. Considering saponification efficiency, a strong alkali is preferable, and further considering also economic efficiency, an aqueous sodium hydroxide solution and an aqueous potassium hydroxide solution are more preferable.

The amount of the aqueous alkaline solution to be brought into contact with the extract is not particularly restricted, and is, for example, 1 vol % or more and 200 vol % or less, preferably 1 vol % or more and 30 vol % or less, and more preferably 1 vol % or more and 10 vol % or less, relative to the total volume of the extract.

The contact of the extract with the aqueous alkaline solution may be conducted in any one of a batch manner and a continuous manner. Industrially, the contact in a continuous manner is preferable in terms of productivity, and the contact in a co-current type continuous manner is particularly preferable in view of cleaning. The stirring time in the contact in a batch manner is not particularly restricted, and is usually 1 minute or more. The average residence time in the case of continuous extract is not particularly restricted, and is usually 10 seconds or more.

The extract after the contact with the aqueous alkaline solution is likely to suffer decomposition of coenzyme Q10 due to heat or the like and deterioration in the quality due to formation of a dimer, and therefore is preferably washed with water. The amount of water to be brought into contact with the extract is not particularly restricted, and is 1 vol % or more and 200 vol % or less, preferably 1 vol % or more and 30 vol % or less, and more preferably 1 vol % or more and 10 vol % or less, relative to the total volume of the extract.

The above contact may be conducted in any one of a batch manner and a continuous manner. Industrially, the contact in a continuous manner is preferable in terms of productivity, and the contact in a co-current type continuous manner is particularly preferable in view of cleaning. The stirring time in the contact in a batch manner is not particularly restricted, and is usually 1 minute or more. The average residence time in the case of continuous extract is not particularly restricted, and is usually 10 seconds or more.

In the production method of the present invention, by the above-described procedures, a microorganism cell suspension containing a coenzyme Q10-producing microorganism that has been concentrated is concentrated with the porous membrane to prepare a concentrated suspension, then a microorganism cell homogenate, an aqueous suspension of the microorganism cell homogenate, dried microorganism cells, or a dried microorganism cell homogenate are prepared from the concentrated suspension as needed, and coenzyme Q10 is extracted in an organic solvent from the concentrated suspension or the microorganism cell homogenate, aqueous suspension of the microorganism cell homogenate, dried microorganism cells, or dried microorganism cell homogenate, and further brought into contact with an aqueous alkaline solution or water as needed. Thereby, coenzyme Q10 that is purified or has improved purity can be separated and recovered.

The coenzyme Q10 solution after washing with water can be directly used or may be purified by further removing impurities using an adsorbent or the like to prepare a coenzyme Q10 extract, and the coenzyme Q10 extract may be further treated to obtain a coenzyme Q10-containing composition or a coenzyme Q10 crystal having a higher purity, which is a more preferred embodiment. Such a treatment step is exemplified by concentration, solvent exchange, oxidation, reduction, column chromatography, crystallization, and a combination thereof. For example, a solvent may be distilled away (for concentration) from the coenzyme Q10 extract which has been separated from the adsorbent to obtain purified coenzyme Q10. Alternatively, after coenzyme Q10 is further purified by column chromatography using silica gel or the like as needed, an organic solvent may be distilled away to obtain purified coenzyme Q10. Furthermore, the target coenzyme Q10 may be obtained as a crystal by crystallization procedure.

Before the above-described column chromatography, oxidation, reduction and crystallization, a solvent may be exchanged as needed.

In the production method of the present invention, in order to produce only reduced coenzyme Q10 or coenzyme Q10 of which reduced coenzyme Q10 ratio is high, a method is effective in which a microorganism which can produce coenzyme Q10 having high reduced coenzyme Q10 ratio is used as the coenzyme Q10-producing microorganism, and an extraction treatment and a purification treatment are conducted after the above-described concentration step under an atmosphere of oxidation resistance, for example, under an inert atmosphere such as nitrogen gas. By this method, only reduced coenzyme Q10 or coenzyme Q10 of which reduced coenzyme Q10 ratio is high can be obtained without special treatment. It is also possible that a reduced coenzyme Q10 ratio can be further increased by reducing the thus obtained coenzyme Q10 having high reduced coenzyme Q10 ratio. In addition, it is also possible that the coenzyme Q10-containing extract is not especially subjected to antioxidant means or is oxidized by oxygen in the air and an oxidant to obtain the coenzyme Q10-containing extract having relatively low reduced coenzyme Q10 ratio such as not more than 50 mol % or not more than 30 mol %, and the obtained coenzyme Q10-containing extract having relatively low reduced coenzyme Q10 ratio is subjected to a reduction reaction, thereby producing coenzyme Q10 having high reduced coenzyme Q10 ratio.

For the purpose of producing reduced coenzyme Q10, it is preferred that a reduced coenzyme Q10 ratio in the final production step or of the final product be high. The reduced coenzyme Q10 ratio in a total amount 100 mol % of coenzyme Q10 may be, for example, 70 mol % or more, preferably 80 mol % or more, more preferably 90 mol % or more, and even more preferably 96 mol % or more.

As the more specific one embodiment, a culture suspension of a coenzyme Q10-producing microorganism is passed through a porous membrane to concentrate, and from the thereby obtained concentrate or the homogenate thereof, coenzyme Q10 is extracted in an organic solvent. The obtained extract containing coenzyme Q10 is further purified by column chromatography and then subjected to a reduction treatment, and highly pure reduced coenzyme Q10 can be obtained as a crystal by a crystallization procedure.

The production method of the present invention can be also used for producing oxidized coenzyme Q10. In such a case, the coenzyme Q10 having high oxidized coenzyme Q10 ratio can be obtained by a simple procedure. For example, coenzyme Q10 is extracted in an organic solvent from a microorganism cell concentrated suspension obtained by concentrating the suspension of a coenzyme Q10-producing microorganism, a microorganism cell homogenate, an aqueous suspension of the microorganism cell homogenate, dried microorganism cells, or a dried microorganism cell homogenate, and the thus obtained extract may be subjected to an oxidation treatment by an oxidant. Alternatively, the coenzyme Q10 having high oxidized coenzyme Q10 ratio can be obtained due to natural oxidation by merely conducting extraction, adsorption, other purification and aftertreatment in the air or drying the microorganism cells in the air before the extraction.

As the more specific one embodiment, a culture suspension of a coenzyme Q10-producing microorganism is passed through a porous membrane to concentrate, and from the thereby obtained concentrate or the homogenate thereof, coenzyme Q10 is extracted in an organic solvent. The obtained extract containing coenzyme Q10 is further purified by column chromatography after the exchange of the solvent and then subjected to an oxidation treatment, and highly pure oxidized coenzyme Q10 may be obtained as a crystal by a crystallization procedure.

The present application claims the benefit of the priority date of Japanese patent application No. 2018-087146 filed on Apr. 27, 2018. All of the contents of the Japanese patent application No. 2018-087146 filed on Apr. 27, 2018, are incorporated by reference herein.

### EXAMPLES

Hereinafter, the present invention is described in more detail with Examples and Comparative Examples but is not restricted to the following Examples. In addition, the yield and purity of coenzyme Q10 in Examples and Comparative Examples do not represent the limiting value of the present invention nor the upper limit.

The concentration of coenzyme Q10 was measured by high-performance liquid chromatography (HPLC) (manufactured by SHIMADZU) in the following condition. (HPLC measurement condition)
Column: YMC-Pack ODS-A
Oven temperature: 30° C.
Mobile phase: methanol/hexane=85/15 by volume
Flow rate: 1.0 ml/min
Detection: UV 275 nm

The concentration degree at the time of filtration with the porous membrane was calculated by directly calculating the amount of the filtrate or measuring the concentration of coenzyme Q10 in the suspension of the coenzyme Q10-producing microorganism under the above HPLC analysis conditions. In addition, the occurrence of loss of coenzyme Q10 was evaluated by measuring the concentration of coenzyme Q10 in the filtrate.

In regard to the washing recovery performance of the porous membrane, on the basis of a permeation rate when 3 hours elapsed since water was passed through the porous membrane before the above suspension of the coenzyme Q10-producing microorganism was passed through, a recovery rate was calculated from a permeation rate when 3 hours elapsed since water was likewise passed through the porous membrane after washing with warm water or chemical agents after the filtration treatment.

### EXAMPLE 1

Saitoella complicata IFO10748 strain, which could produce coenzyme Q10, was aerobically cultivated in a culture medium (peptone 5 g/L, yeast extract 3 g/L, malt extract 3 g/L, glucose 20 g/L, pH 6.0) at 25° C. for 160 hours.

Thereafter, the obtained microorganism culture medium containing coenzyme Q10 was heated to 60° C., adjusted to pH 6, and then passed through a porous membrane (Φ=6 mm, L=609 mm, average pore size=0.5 μm, filtration area=0.0114 m²; manufactured by Graver) including a hollow porous body made of titanium oxide and a support made of stainless steel at a linear velocity of 4 m/s and a transmembrane pressure difference (TMP) of 0.2 MPa, and thus a filtration treatment was performed.

As a result of continuous operation for 10 hours, the microorganism culture medium, of which solid concentration (corresponding to a microorganism concentration in terms of dry weight of the microorganism in the microorganism cell suspension) before the filtration treatment was 8.06%, was concentrated, and the solid concentration after the filtration treatment was 10.35%. The average permeation flux throughout the filtration step was 0.69 kg/min/m².

The concentration of coenzyme Q10 in the filtrate was measured and found to be below the limits of detection.

Furthermore, the microorganism in the obtained microorganism concentrated suspension was homogenized by a pressure homogenizer. To the obtained microorganism homogenate were added 1.8 times amount of hexane and 0.7 times amount of 2-propanol to the volume of the microorganism homogenate, and the resulting mixture was stirred at 40° C. for 1 hour. This operation was repeated twice to extract coenzyme Q10 by a two-stage batch extraction operation. As a result, the extraction rate was 96.8%, and it was confirmed that coenzyme Q10 was satisfactorily extracted by concentration with the porous membrane.

### EXAMPLE 2

A microorganism culture medium containing coenzyme Q10 obtained in the same manner as in Example 1 was heated to 50° C., adjusted to pH 6, and then passed through the same porous membrane as that in Example 1 at a linear velocity of 3 m/s and a transmembrane pressure difference (TMP) of 0.3 MPa, and thus a filtration treatment was performed.

As a result of continuous operation for 10 hours, the microorganism culture medium, of which solid concentration before the filtration treatment was 8.06%, was concentrated, and the solid concentration after the filtration treatment was 10.32%. The average permeation flux throughout the filtration step was 0.59 kg/min/m².

The concentration of coenzyme Q10 in the filtrate was measured and found to be below the limits of detection.

Furthermore, the obtained microorganism concentrated suspension was subjected to pressure homogenization in the same manner as in Example 1 to extract coenzyme Q10. As a result, the extraction rate was 97.1%, and it was confirmed that coenzyme Q10 was satisfactorily extracted by concentration with the porous membrane.

### EXAMPLE 3

A microorganism culture medium containing coenzyme Q10 obtained in the same manner as in Example 1 was heated to 40° C., adjusted to pH 6, and then passed through the same porous membrane as that in Example 1 at a linear velocity of 5 m/s and a transmembrane pressure difference (TMP) of 0.4 MPa, and thus a filtration treatment was performed.

As a result of continuous operation for 11.2 hours, the microorganism culture medium, of which solid concentration before the filtration treatment was 8.06%, was concentrated, and the solid concentration after the filtration treatment was 10.85%. The average permeation flux throughout the filtration step was 0.78 kg/min/m².

The concentration of coenzyme Q10 in the filtrate was measured and found to be below the limits of detection.

Furthermore, the obtained microorganism concentrated suspension was subjected to pressure homogenization in the same manner as in Example 1 to extract coenzyme Q10. As a result, the extraction rate was 97.0%, and it was confirmed that coenzyme Q10 was satisfactorily extracted by concentration with the porous membrane.

### EXAMPLE 4

A microorganism culture medium containing coenzyme Q10 obtained in the same manner as in Example 1 was heated to 60° C., adjusted to pH 5, and then passed through the same porous membrane as that in Example 1 at a linear velocity of 3 m/s and a transmembrane pressure difference (TMP) of 0.4 MPa, and thus a filtration treatment was performed.

As a result of continuous operation for 10 hours, the microorganism culture medium, of which solid concentration before the filtration treatment was 8.06%, was concentrated, and the solid concentration after the filtration treatment was 10.27%. The average permeation flux throughout the filtration step was 0.55 kg/min/m².

The concentration of coenzyme Q10 in the filtrate was measured and found to be below the limits of detection.

Furthermore, the obtained microorganism concentrated suspension was subjected to pressure homogenization in the same manner as in Example 1 to extract coenzyme Q10. As a result, the extraction rate was 94.6%, and it was confirmed that coenzyme Q10 was satisfactorily extracted by concentration with the porous membrane.

### EXAMPLE 5

A microorganism culture medium containing coenzyme Q10 obtained in the same manner as in Example 1 was heated to 50° C., adjusted to pH 4, and then passed through the same porous membrane as that in Example 1 at a linear velocity of 4 m/s and a transmembrane pressure difference (TMP) of 0.4 MPa, and thus a filtration treatment was performed.

As a result of continuous operation for 8.5 hours, the microorganism culture medium, of which solid concentration before the filtration treatment was 8.06%, was concentrated, and the solid concentration after the filtration treatment was 11.0%. The average permeation flux throughout the filtration step was 1.09 kg/min/m².

The concentration of coenzyme Q10 in the filtrate was measured and found to be below the limits of detection.

Furthermore, the obtained microorganism concentrated suspension was subjected to pressure homogenization in the same manner as in Example 1 to extract coenzyme Q10. As a result, the extraction rate was 97.2%, and it was confirmed that coenzyme Q10 was satisfactorily extracted by concentration with the porous membrane.

### EXAMPLE 6

A microorganism culture medium containing coenzyme Q10 obtained in the same manner as in Example 1 was heated to 60° C., adjusted to pH 4, and then passed through the same porous membrane as that in Example 1 at a linear velocity of 5 m/s and a transmembrane pressure difference (TMP) of 0.3 MPa, and thus a filtration treatment was performed.

As a result of continuous operation for 6 hours, the microorganism culture medium, of which solid concentration before the filtration treatment was 8.15%, was concentrated, and the solid concentration after the filtration treatment was 13.04%. The average permeation flux throughout the filtration step was 1.60 kg/min/m².

The concentration of coenzyme Q10 in the filtrate was measured and found to be below the limits of detection.

Furthermore, the obtained microorganism concentrated suspension was subjected to pressure homogenization in the same manner as in Example 1 to extract coenzyme Q10. As a result, the extraction rate was 96.9%, and it was confirmed that coenzyme Q10 was satisfactorily extracted by concentration with the porous membrane.

### EXAMPLE 7

A microorganism culture medium containing coenzyme Q10 obtained in the same manner as in Example 1 was heated to 40° C., adjusted to pH 4, and then passed through the same porous membrane as that in Example 1 at a linear velocity of 3 m/s and a transmembrane pressure difference (TMP) of 0.2 MPa, and thus a filtration treatment was performed.

As a result of continuous operation for 10 hours, the microorganism culture medium, of which solid concentration before the filtration treatment was 8.06%, was concentrated, and the solid concentration after the filtration treatment was 10.09%. The average permeation flux throughout the filtration step was 0.65 kg/min/m².

The concentration of coenzyme Q10 in the filtrate was measured and found to be below the limits of detection.

Furthermore, the obtained microorganism concentrated suspension was subjected to pressure homogenization in the same manner as in Example 1 to extract coenzyme Q10. As a result, the extraction rate was 96.9%, and it was confirmed that coenzyme Q10 was satisfactorily extracted by concentration with the porous membrane.

### EXAMPLE 8

A microorganism culture medium containing coenzyme Q10 obtained in the same manner as in Example 1 was heated to 50° C., adjusted to pH 5, and then passed through the same porous membrane as that in Example 1 at a linear velocity of 5 m/s and a transmembrane pressure difference (TMP) of 0.2 MPa, and thus a filtration treatment was performed.

As a result of continuous operation for 8.7 hours, the microorganism culture medium, of which solid concentration before the filtration treatment was 8.06%, was concentrated, and the solid concentration after the filtration treatment was 11.01%. The average permeation flux throughout the filtration step was 1.08 kg/min/m².

The concentration of coenzyme Q10 in the filtrate was measured and found to be below the limits of detection.

Furthermore, the obtained microorganism concentrated suspension was subjected to pressure homogenization in the same manner as in Example 1 to extract coenzyme Q10. As a result, the extraction rate was 95.9%, and it was confirmed that coenzyme Q10 was satisfactorily extracted by concentration with the porous membrane.

### EXAMPLE 9

A microorganism culture medium containing coenzyme Q10 obtained in the same manner as in Example 1 was heated to 50° C., adjusted to pH 4, and then passed through the same porous membrane as that in Example 1 at a linear velocity of 5 m/s and a transmembrane pressure difference (TMP) of 0.2 MPa, and thus a filtration treatment was performed.

As a result of continuous operation for 6.7 hours, the microorganism culture medium, of which solid concentration before the filtration treatment was 7.9%, was concentrated, and the solid concentration after the filtration treatment was 12.2%. The average permeation flux throughout the filtration step was 1.65 kg/min/m².

The concentration of coenzyme Q10 in the filtrate was measured and found to be below the limits of detection.

Furthermore, the obtained microorganism concentrated suspension was subjected to pressure homogenization in the same manner as in Example 1 to extract coenzyme Q10. As a result, the extraction rate was 98.0%, and it was confirmed that coenzyme Q10 was satisfactorily extracted by concentration with the porous membrane.

### EXAMPLE 10

A microorganism culture medium containing coenzyme Q10 obtained in the same manner as in Example 1 was heated to 50° C., adjusted to pH 4, and then passed through a porous membrane (Φ=9 mm, L=1520 mm, average pore size: 0.5 gm, filtration area: 0.0462 m²; manufactured by Graver) including a hollow porous body made of titanium oxide and a support made of stainless steel at a linear velocity of 5 m/s and a transmembrane pressure difference (TMP) of 0.2 MPa, and thus a filtration treatment was performed.

As a result of continuous operation for 29.5 hours, the microorganism culture medium, of which solid concentration before the filtration treatment was 7.11%, was concentrated, and the solid concentration after the filtration treatment was 12.96%. The average permeation flux throughout the filtration step was 2.35 kg/min/m².

The concentration of coenzyme Q10 in the filtrate was measured and found to be below the limits of detection.

Furthermore, the obtained microorganism concentrated suspension was subjected to pressure homogenization in the same manner as in Example 1 to extract coenzyme Q10. As a result, the extraction rate was 98.0%, and it was confirmed that coenzyme Q10 was satisfactorily extracted by concentration with the porous membrane.

### EXAMPLE 11

During the filtration operation of Example 10, air was allowed to flow in from a filtrate discharge side for 30 seconds to perform washing and a regeneration treatment of the porous membrane. As a result, the permeation flux before the regeneration treatment was 1.49 kg/min/m², whereas the permeation flux after the regeneration treatment was restored to 2.34 kg/min/m².

### EXAMPLE 12

After the filtration operation of Example 11, the porous membrane was subjected to circulation washing with warm water of 50° C. for 1 hour. As a result, the washing recovery performance was 40%. The concentration of coenzyme Q10 in the wash liquid used in washing was measured and found to be below the limits of detection.

### EXAMPLE 13

After Example 12, the porous membrane was subjected to circulation washing with a 2% aqueous sodium hydroxide solution of 70° C. for 1 hour. As a result, the washing recovery performance was 97%.

The concentration of coenzyme Q10 in the wash liquid used in washing was measured and found to be below the limits of detection.

### EXAMPLE 14

A microorganism culture medium containing coenzyme Q10 obtained in the same manner as in Example 1 was heated to 50° C., adjusted to pH 5, and then passed through the same porous membrane as that in Example 1 at a linear velocity of 10 m/s and a transmembrane pressure difference (TMP) of 0.25 MPa, and thus a filtration treatment was performed.

As a result of continuous operation for 7.5 hours, the microorganism culture medium, of which solid concentration before the filtration treatment was 6.96%, was concentrated, and the solid concentration after the filtration treatment was 13.29%. The average permeation flux throughout the filtration step was 3.41 kg/min/m².

The concentration of coenzyme Q10 in the filtrate was measured and found to be below the limits of detection.

### EXAMPLE 15

A microorganism culture medium containing coenzyme Q10 obtained in the same manner as in Example 1 was heated to 50° C., adjusted to pH 5, and then passed through the same porous membrane as that in Example 1 at a transmembrane pressure difference (TMP) of 0.35 MPa while changing the linear velocity as follows: 7 m/s for 4 hours, then 5 m/s for 5.5 hours, and further 6 m/s for 4 hours, and thus a filtration treatment was performed.

As a result of continuous operation for 13.5 hours in total, the microorganism culture medium, of which solid concentration before the filtration treatment was 7.46%, was concentrated, and the solid concentration after the filtration treatment was 12.71%. The average permeation flux throughout the filtration step was 2.74 kg/min/m².

The concentration of coenzyme Q10 in the filtrate was measured and found to be below the limits of detection.

### EXAMPLE 16

A microorganism culture medium containing coenzyme Q10 obtained in the same manner as in Example 1 was heated to 50° C., adjusted to pH 5, and then passed through the same porous membrane as that in Example 1 at a linear velocity of 7 m/s and a transmembrane pressure difference (TMP) of 0.25 MPa, and thus a filtration treatment was performed. When confirmed that the solid concentration of 6.71% before the filtration treatment became 12.5%, a continuous repeat test in which part of the obtained filtrate was returned to a stock solution of the microorganism culture suspension before the treatment, and the resulting solution was diluted to a solid concentration of 9.5% and concentrated again to a solid concentration of 12.5% was performed four times in total.

The average permeation flux throughout the time period until each solution was concentrated to the predetermined concentration was 2.41 kg/min/m² in the first test, 2.40 kg/min/m² in the second test, 2.08 kg/min/m² in the third test, and 1.38 kg/min/m² in the fourth test. Although the average permeation flux gradually decreased, the filtration was satisfactorily performed through four repeated operations.

### EXAMPLE 17

A microorganism culture medium containing coenzyme Q10 obtained in the same manner as in Example 1 was heated to 50° C., adjusted to pH 5, and then passed through a ceramic membrane (Φ=3.5 mm, L=1187 mm, average pore size: 0.2 gm, filtration area: 0.35 m²; manufactured by TAMI) at a linear velocity of 7.5 m/s and a transmembrane pressure difference (TMP) of 0.19 MPa, and thus a filtration treatment was performed.

As a result of continuous operation for 1.5 hours, the microorganism culture medium, of which solid concentration before the filtration treatment was 8.0%, was concentrated, and the solid concentration after the filtration treatment was 12.84%. The average permeation flux throughout the filtration step was 2.92 kg/min/m².

The concentration of coenzyme Q10 in the filtrate was measured and found to be below the limits of detection.

### EXAMPLE 18

A microorganism culture medium containing coenzyme Q10 obtained in the same manner as in Example 1 was heated to 50° C., adjusted to pH 5, and then passed through the same ceramic membrane as that in Example 17 at a linear velocity of 7 m/s and a transmembrane pressure difference (TMP) of 0.3 MPa, and thus a filtration treatment was performed.

As a result of continuous operation for 30 minutes, the microorganism culture medium, of which solid concentration before the filtration treatment was 7.57%, was concentrated, and the solid concentration after the filtration treatment was 12.93%. The average permeation flux throughout the filtration step was 3.81 kg/min/m². Then, the operation was switched to a circulation treatment and performed for 20 hours. As a result, the average permeation flux during the circulation treatment dropped to 1.32 kg/min/m², and the average permeation flux throughout the whole operation was 1.80 kg/min/m².

### Comparative Example 1

Using a Kiriyama funnel and No. 5-C filter paper for use in the Kiriyama funnel, an attempt was made to filter a microorganism culture medium containing coenzyme Q10 (solid concentration: 8.06%) obtained in the same manner as in Example 1, but clogging occurred from the beginning, so that no filtrate was obtained.

### Comparative Example 2

A microorganism culture medium containing coenzyme Q10 (solid concentration: 8.06%) obtained in the same manner as in Example 1 was centrifuged at 1000 g for 5 minutes with Allegra X-22R CENTRIGUGE manufactured by Beckman Coulter to separate into a microorganism concentrate and a supernatant, and the supernatant was collected. The concentration of coenzyme Q10 in the supernatant was 0.3 g/L, and it was confirmed that 1.4% of coenzyme Q10 in the microorganism culture medium was lost.

### Comparative Example 3

A microorganism culture medium containing coenzyme Q10 (solid concentration: 8.06%) obtained in the same manner as in Example 1 was centrifuged at 2000 g for 5 minutes with the same centrifugal separator as that in Comparative Example 2 to separate into a microorganism concentrate and a supernatant, and the supernatant was collected. The concentration of coenzyme Q10 in the supernatant was 0.1 g/L, and it was confirmed that 0.6% of coenzyme Q10 in the microorganism culture medium was lost.

### Comparative Example 4

A microorganism culture medium containing coenzyme Q10 obtained in the same manner as in Example 1 was heated to 30° C., adjusted to pH 5, and then passed through a ceramic membrane (Φ=6 mm, L=1187 mm, average pore size: 0.2 μm, filtration area: 0.16 m²; manufactured by TAMI) at a linear velocity of 3 m/s and a transmembrane pressure difference (TMP) of 0.05 MPa, and thus a filtration treatment was performed.

As a result of continuous operation for 4.5 hours, the microorganism culture medium, of which solid concentration before the filtration treatment was 7.0%, was concentrated, and the solid concentration after the filtration treatment was 11.72%. The average permeation flux throughout the filtration step was as rather low as 0.49 kg/min/m².

### Comparative Example 5

A microorganism culture medium containing coenzyme Q10 obtained in the same manner as in Example 1 was heated to 30° C., adjusted to pH 5, and then passed through a organic membrane (average pore size: 0.2 μm, filtration area: 0.022 m²; manufactured by DAISEN) at a linear velocity of 1.1 m/s and a transmembrane pressure difference (TMP) of 0.015 MPa, and thus a filtration treatment was performed.

As a result of continuous operation for 12 hours, the microorganism culture medium, of which solid concentration before the filtration treatment was 6.75%, was concentrated, and the solid concentration after the filtration treatment was 9.98%. The average permeation flux throughout the filtration step was as rather low as 0.42 kg/min/m².

1. A method for producing coenzyme Q10 comprising:
filtering a culture suspension of a coenzyme Q10-producing microorganism by passing the culture suspension through a porous membrane, wherein the culture suspension is heated to a temperature of 35° C. or higher.

2. The method according to claim 1, wherein the culture suspension is heated to a temperature of
48° C. or higher.

3. The method according to claim 1, wherein the culture suspension has a pH in a range of 3 to 7.

4. The method according to claim 1, wherein the filtering is conducted with
a linear velocity of the culture suspension of 0.1 m/s or more.

5. The method according to claim 1, wherein:
the filtering further comprises a regenerating step of closing a filtration device, applying a pressure to the filtration device, and then releasing the pressure, and
wherein the regenerating step is performed at least once.

6. The method according to claim 5, wherein the pressure applied to the filtration device is 0.1 to 1 MPa.

7. The method according to claim 5, wherein: a medium for applying pressure is at least one of air, nitrogen, water, a second culture suspension and a filtrate obtained by passing the culture suspension through the porous membrane.

8. The method according to claim 1, further comprising, after a completion of the filtering:
washing a microorganism suspended component that was adhered to the porous membrane, and then,
performing a second filtering step.

9. The method according to claim 8, wherein the washing uses
a wash liquid comprising at least one of water, an aqueous alkaline solution, and an aqueous acidic solution.

10. The method according to claim 9, wherein
a temperature of the wash liquid is from 10° C. to 90° C.

11. The method according to claim 1, wherein
the porous membrane is made of a synthetic resin, ceramic, or metal.

12. The method according to claim 11, wherein
the porous membrane is made of metal.

13. The method according to claim 12, wherein,
the porous membrane made of metal comprises a separation layer of titanium, oxide; and
the separation layer is supported by a cylindrical support of stainless steel with an inner diameter of 5 mm or more.

14. The method according to claim 13, wherein
the separation layer has an average pore size of 0.01 to 3 μm.

15. The method according to claim 1, wherein the filtering further comprises:
a treatment step of allowing a medium to flow in from a filtrate outlet side and passing the medium through the porous membrane; and
performing an additional filtering step.

16. The method according to claim 15, wherein,
the medium that is allowed to flow in is at least one of air, nitrogen, water, and
a filtrate obtained by passing the culture suspension through the porous membrane.

17. The method according to claim 15, wherein the medium that is allowed to flow is at
a temperature of 10° C. to 90° C.
